(19) United States
(12) Patent Application Publication
(10) Pub. No.: US 2026/0345385 A1
(43) Pub. Date: Nov. 5, 2026

(54) MOTORIZED RETINAL TRANSPLANT DELIVERY DEVICE AND METHOD OF USE

(71) Applicant: The Regents of the University of California, Oakland, CA (US)

(72) Inventors: Andrew BROWNE, Irvine, CA (US); Magdalene SEILER, Irvine, CA (US); William TANG, Irvine, CA (US); Francis UNG, Irvine, CA (US)

(73) Assignee: The Regents of the University of California, Oakland, CA (US)

(21) Appl. No.: 19/666,488

(22) Filed: May 4, 2026

Related U.S. Application Data
(60) Provisional application No. 63/799,731, filed on May 4, 2025.
(60) Provisional application No. 63/830,029, filed on Jun. 25, 2025.

(51) Int. Cl. A61F 9/007

(52) U.S. Cl. CPC A61F 9/00736

(57) ABSTRACT

Retinal transplantation holds potential for vision restoration in severe retinal degeneration, but manual delivery of retinal sheets often suffers from imprecision and tissue damage due to unstable instruments. To address this, researchers developed a 3D-printed, motorized retinal sheet injector controlled via Arduino and a foot pedal with three preset actuator positions. In comparative tests with four participants, motion analysis showed that the manual injector caused nearly three times more tip movement variance than the motorized version (p<10⁻¹¹). Although tip velocities were generally lower with the motorized injector, the differences were not statistically significant. In placement accuracy trials using gelatin models, the motorized system significantly outperformed the manual one (~52% vs. ~37% accuracy, p=0.00999), with complete misplacements occurring only in the manual group.

### CROSS-REFERENCE TO RELATED APPLICATIONS

This application claims the benefit of U.S. provisional application Ser. No. 63/799,731 filed May 4, 2025 and U.S. provisional application Ser. No. 63/830,029 filed Jun. 25, 2025, the disclosures of which are hereby incorporated in their entirety by reference herein.

### STATEMENT REGARDING FEDERALLY SPONSORED RESEARCH OR DEVELOPMENT

The invention was made with Government support under Contract Nos. EY034912; EY031834; EY032948; and EY04070 awarded by the National Institutes of Health. The Government has certain rights to the invention.

### TECHNICAL FIELD

In at least one aspect, the disclosure provides a motorized retinal sheet injector system configured to enable precise and stable subretinal delivery of retinal tissue grafts, such as retinal organoids or sheets, by incorporating a foot pedal-controlled actuator mechanism that minimizes operator-induced motion artifacts and improves placement accuracy relative to manual delivery methods.

### BACKGROUND

Retinal degeneration in diseases like age-related macular degeneration (AMD), and retinitis pigmentosa—results in irreversible loss of photoreceptors or retinal pigment epithelium (RPE), ultimately leading to blindness. Although early intervention is crucial to preserve vision, there are currently no broadly available therapies that halt progression during the early stages of these diseases. Therefore, advanced disease with cell death and vision loss remains common.

In advanced retinal degeneration, synthetic retinal stimulation offers a means to restore limited visual function. Bioelectronic prosthetics, such as the Argus II retinal prosthesis (Second Sight Medical Products, Inc., Sylmar, CA, USA), electrically stimulate surviving inner retinal neurons to evoke rudimentary visual perceptions.¹ Several groups have demonstrated the potential of devices that electrically stimulate the retina.²⁻⁴ While bioelectronic prosthetics can elicit basic visual responses, they remain limited in their spatial resolution and non-physiological stimulation patterns.

Cell replacement therapy aims to restore retinal function by replacing lost cells through various strategies: transplantation of dissociated cells, cell sheets, or intact retinal tissue. For example, RPE transplantation has been performed by subretinal injection of dissociated human embryonic stem cell (hESC)-derived RPE in patients with AMD⁵ and by transvitreal implantation of hESC-RPE on an ultrathin synthetic parylene substrate designed to mimic Bruch's membrane⁶, with the latter approach reporting only mild, self-resolving adverse events. Other approaches include the transplantation of dissociated photoreceptors and the use of fetal retinal tissue or retinal organoid sheets, which have demonstrated functional integration in preclinical studies.⁷⁻¹⁷

Despite these advances, achieving precise delivery of retinal tissue without collateral damage to the host tissue remains challenging. To improve placement precision, Aramant and Seiler developed a patented (Patent ID: 8057483) instrument that gently places the retinal sheet into the subretinal space, reducing the risk of increased intraocular pressure from excess fluid.^{8,9} The first-generation retinal sheet transplantation instrument (FIG. 1A) was designed with pencil like handle with a stationary, flat mandrel extending from the handle to the instrument's tip. A polytetrafluoroethylene tip was manually fashioned from heat shrink tubing over a mandrel mold and placed over the mandrel like a sock with an opening at the end. A thumb-controlled lever with a spring was used to manipulate the position of the nozzle over fixed mandrel. Operation of the instrument (FIG. 1B) involved placing a sheet of retinal tissue in front of the mandrel tip while the nozzle was moved from a retracted position to advance over the retinal sheet. The thumb-controlled manipulator was then locked into place with a clasp. To inject the retinal sheet into the subretinal space, the loaded nozzle was placed into the subretinal space, the thumb-controlled lever was unlocked by delicately squeezing the manipulator bar, and the nozzle was retracted over the retinal sheet by again delicately releasing all the spring tension applied to the manipulator bar, while the mandrel remained fixed in place to hold the retinal sheet in place. The process of manually unlocking and releasing the spring-loaded bar is nearly impossible to perform without small movements by the operator's hand, and small movements on the handle result in larger movements at the instrument tip. This uncontrollable movement can affect transplant placement precision and overall success due to potential damage to surrounding tissues.

While their method successfully facilitated the replacement of damaged photoreceptors with functional ones from hPSC-derived retinal organoids and led to some visual recovery in retinal degenerative models, a significant number of transplants still resulted in rosette formation complications. Additionally, improper positioning of the transplant within the vitreous by the user further contributed to this issue, accentuating the need for an automated instrument capable of highly precise movements and accurate graft placement.

Accordingly, there is a need for a motorized retinal transplant delivery device that improves placement precision and reduces operator-induced motion artifacts during subretinal sheet transplantation.

### SUMMARY

In at least one aspect, to address the unmet needs set forth above, a motorized injector was developed with the goals of reducing production costs, enhancing the accuracy of retinal sheet placement, and minimizing complications and collateral tissue damage. A further objective of the design was to allow for fabrication using off-the-shelf components that are compatible with 3D printing and simple machining processes.

In another aspect, a motorized retinal sheet injector for delivering retinal material into the subretinal space is provided. The motorized retinal sheet injector includes a handheld injector housing, a linear actuator at least partially housed within the injector housing, and a mandrel fixed relative to the injector housing and extending along a longitudinal axis. The injector further includes a core shaft, a magnetic coupler operably connecting the linear actuator to the core shaft, and a mandrel anchor point fixed to the core shaft. A nozzle is disposed coaxially with the mandrel and is configured to slide along the mandrel when actuated, the nozzle having a nozzle tip. Additionally, a nozzle coupler connects the mandrel anchor point to the nozzle such that actuation force from the linear actuator is transmitted to the nozzle via the core shaft. The injector further comprises a microcontroller in electrical communication with the linear actuator and a foot pedal interface in electrical communication with the microcontroller. The microcontroller is configured to control the actuator based on input from the foot pedal interface, positioning the nozzle in at least three discrete positions: a retracted position (“Min”), an intermediate position (“Mid”), and a fully extended position (“Max”).

In another aspect, a method for delivering retinal material into the subretinal space of an eye is provided. The method includes a step of providing a motorized retinal sheet injector system. A retinal sheet is loaded into the nozzle. A distal end of the injector is inserted into a subretinal space of an eye of the subject. A foot pedal is actuated to advance the nozzle over the retinal sheet via the actuator, magnetic coupler, and core shaft assembly, releasing the retinal sheet into the subretinal space. Finally, the injector is withdrawn while maintaining the mandrel in a fixed position.

The foregoing summary is illustrative only and is not intended to be in any way limiting. In addition to the illustrative aspects, features described above, further aspects, features will become apparent by reference to the drawings and the following detailed description.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements and wherein:

FIGS. 1A and 1B: Manual injector (A) and (B) operating principle for transplanting sheets of retinal tissue into the subretinal space.

FIGS. 2A, 2B, 2C, and 2D: Design of motorized injector and system. (A) Exploded View of the Actuation Assembly. (B) Nozzle Actuation Mechanism. Small movements of the linear actuator, transmitted through the magnetic coupler, displace the nozzle coupler and nozzle over a stationary mandrel. (C) Schematic of fully Assembled Injector System. The assembled injector features three actuator pedals controlled by an Arduino, achieving minimum, middle, and maximum nozzle positions. (D) Wiring diagram for Arduino microcontroller.

FIGS. 3A, 3B, and 3C: Renderings from photographs of motorized subretinal injector system with (A) disassembled components of the handheld motorized injector (B) the assembled injector, and (C) fully assembled system.

FIGS. 4A, 4B, 4C, and 4D: The tips of the injectors were tracked and the positions plotted on a cartesian plane. (A) represents the manual injector and (B) represents the motorized injector. The pathways of each participant are plotted on the same graph. Average tip velocity (C) and Maximum tip velocity (D) for each participant are represented on a box plot and separated by type of injector.

FIGS. 5A, 5B, and 5C: Each participant was instructed to place an injectable piece of paper over a landing strip. The % Target overlap was then calculated by calculating the area of the paper overlapping with the landing strip with the total area of the injectable. (A) A single trial of the injectable over the target landing strip. (B) Dark coloring was applied to highlight the overlapping areas-the area of the overlapping area was calculated then divided by the total area of the injectable to achieve % target overlap. (C) Percent target overlap for each trial represented in a box plot for manual vs automated injector.

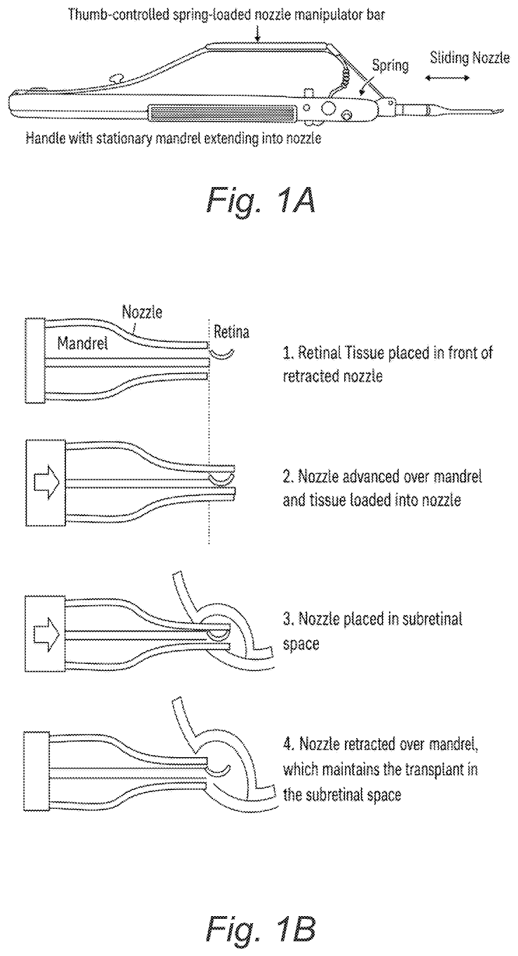

### DETAILED DESCRIPTION

Reference will now be made in detail to presently preferred aspects and methods of the present invention, which constitute the best modes of practicing the invention presently known to the inventors. The Figures are not necessarily to scale. However, it is to be understood that the disclosed aspects are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific aspects and methods described below, as specific components and/or conditions may, of course, vary. Furthermore, the terminology used herein is used only for the purpose of describing particular aspects of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

When referring to a numerical quantity, in a refinement, the term “less than” includes a lower non-included limit that is 5 percent of the number indicated after “less than.” A lower non-includes limit means that the numerical quantity being described is greater than the value indicated as a lower non-included limited. For example, “less than 20” includes a lower non-included limit of 1 in a refinement. Therefore, this refinement of “less than 20” includes a range between 1 and 20. In another refinement, the term “less than” includes a lower non-included limit that is, in increasing order of preference, 20 percent, 10 percent, 5 percent, 1 percent, or 0 percent of the number indicated after “less than.”

The term “one or more” means “at least one” and the term “at least one” means “one or more.” The terms “one or more” and “at least one” include “plurality” as a subset.

The term “substantially,” “generally,” or “about” may be used herein to describe disclosed or claimed aspects. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

Throughout this application, where publications are referenced, the disclosures of these publications in their entireties are hereby incorporated by reference into this application to more fully describe the state of the art to which this invention pertains.

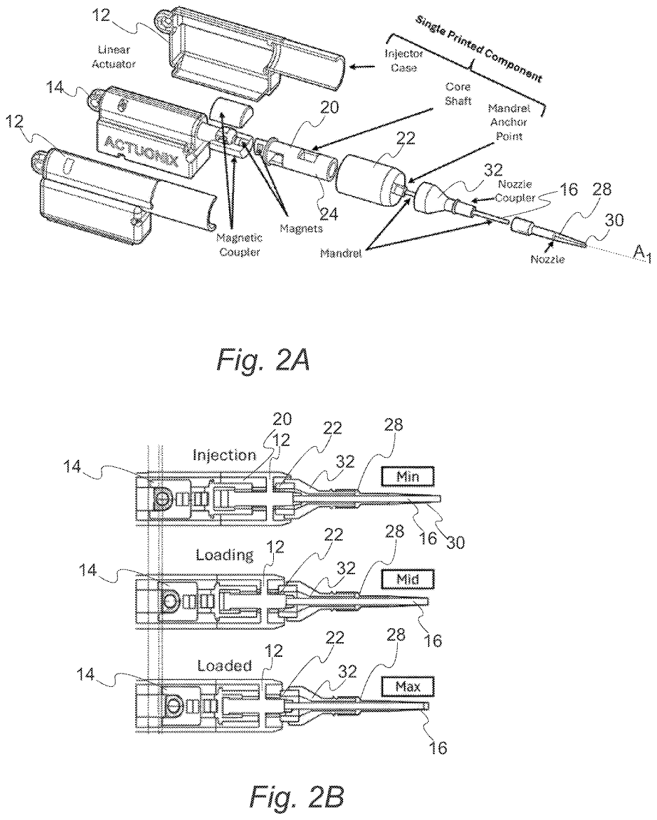

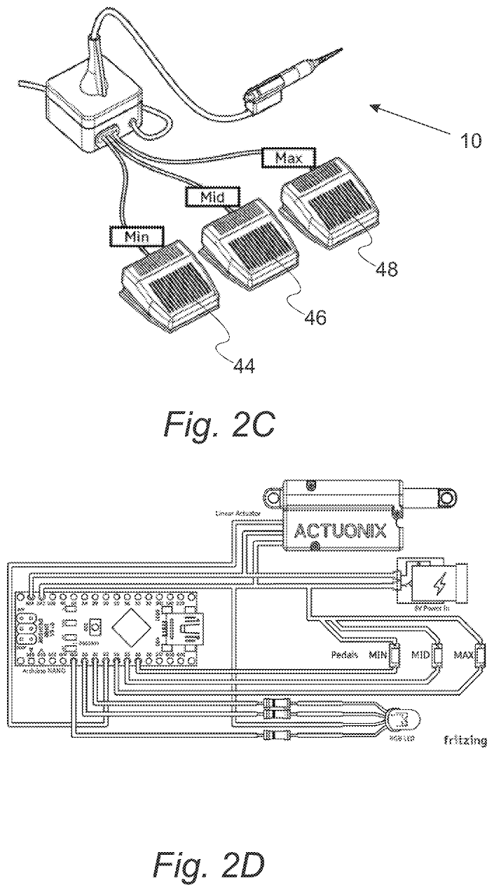

Referring to FIGS. 2A and 2B, a schematic of a motorized retinal sheet injector for delivering retinal material into the subretinal space is provided. The material can be a sheet of retinal tissue, matrix of cells or tissue, a synthetic object, a drug depot, a gene therapy depot, or some composite thereof. The material can be a liquid bolus occupying nozzle tip volume. The motorized retinal sheet injector 10 includes a handheld injector housing 12 and a linear actuator 14 at least partially housed within the handheld injector housing 12. A mandrel 16 fixed relative to the handheld injector housing 12 and extending along a longitudinal axis A1. For example, mandrel 16 can be fixed to a structure in or on the housing (e.g., a rib). A core shaft 20 is attached to the linear actuator 14. A magnetic coupler 22 operably connects the linear actuator 14 to the core shaft 20. A mandrel anchor point 24 is fixed to the core shaft 20. A nozzle 28 is disposed coaxially with the mandrel 16 and configured to slide along the mandrel when actuated. The nozzle 28 has a nozzle tip 30. A nozzle coupler 32 is configured to connect the mandrel anchor point 24 to the nozzle 28, such that actuation force from the linear actuator 14 is transmitted to the nozzle via the core shaft 20. A microcontroller 36 is provided in electrical communication with the linear actuator 14. A foot pedal interface in electrical communication with the microcontroller and foot pedals 44, 46, 48. The microcontroller 36 is configured to control the linear actuator based on input from the foot pedal interface. The linear actuator 14 is operable to position the nozzle in at least three discrete positions including a retracted position (“Min”), an intermediate position (“Mid”), and a fully extended position (“Max”).

In another aspect, at least one of the handheld injector housing 12, the magnetic coupler 22, mandrel anchor point 24, the nozzle 28, and the nozzle coupler 32 is at least partially fabricated using a 3D printing process with a biocompatible, sterilizable resin. In a refinement, the nozzle 28 includes fluorinated ethylene propylene (FEP) heat-shrink tubing conformally fitted over the mandrel to provide a sleeve for retaining the retinal sheet. In a refinement, the mandrel 16 is made of stainless steel to provide structural rigidity and biocompatibility.

In another aspect, the injector is further configured for loading a retinal sheet into the nozzle. The microcontroller maintains the nozzle in a retracted (“Min”) position while the retinal sheet is positioned on a sterile working surface under visual magnification, advances the nozzle to an intermediate (“Mid”) position to align the nozzle tip with the retinal sheet, and further advances the nozzle toward the fully extended position (“Max”) to engage and envelop the retinal sheet within the nozzle without compressing it.

In another aspect, the microcontroller includes a microcontroller board configured to receive foot pedal inputs and actuate the linear actuator to the Min, Mid, and Max positions.

In another aspect, a method for treating a subject with retinal degeneration is provided. The method includes a step of providing a motorized retinal sheet injector system. A retinal sheet is loaded into the nozzle. A distal end of the injector is inserted into a subretinal space of an eye of the subject. A foot pedal is actuated to advance the nozzle over the retinal sheet via the actuator, magnetic coupler, and core shaft assembly, releasing the retinal sheet into the subretinal space. Finally, the injector is withdrawn while maintaining the mandrel in a fixed position.

In another aspect, the retinal sheet includes a retinal organoid derived from human pluripotent stem cells, which can include human embryonic stem cells (hESCs) or induced pluripotent stem cells (iPSCs). These retinal organoids may exhibit organized cellular layers reminiscent of natural retinal architecture, containing differentiated photoreceptor cells and retinal pigment epithelial cells capable of functional integration and visual restoration upon transplantation into the subretinal space.

In another aspect, the retinal degeneration condition treated by the methods disclosed herein is selected from age-related macular degeneration (AMD), retinitis pigmentosa (RP), and Stargardt disease, encompassing conditions characterized by progressive loss of photoreceptor cells and impairment of visual function. The methods described are particularly beneficial for addressing retinal degeneration stages where conventional treatments have limited efficacy, thus providing a potentially transformative therapeutic approach.

In another aspect, the process of loading the retinal sheet into the nozzle includes positioning the retinal sheet on a sterile, microscopically observable surface, carefully orienting the retinal sheet to achieve the desired cellular polarity and structural orientation, partially advancing the nozzle to an intermediate “Mid” position to facilitate capture, gently and precisely drawing the retinal sheet into the nozzle tip without deformation or damage, and meticulously verifying the central alignment and proper positioning of the retinal sheet within the nozzle prior to subretinal injection. This precise method ensures optimal orientation and integrity of the retinal sheet, enhancing the potential for successful graft integration and function upon transplantation.

In another aspect, the nozzle includes a fluorinated ethylene propylene (FEP) sleeve that is precisely heat-conformed around the mandrel. This heat-conforming process ensures a tailored, snug fit of the sleeve, providing optimal mechanical properties, biocompatibility, and a smooth internal surface to facilitate gentle and controlled handling and delivery of the retinal sheet into the subretinal space, thereby minimizing tissue stress and potential complications during transplantation.

In another aspect, the foot pedal interface enables precise, hands-free selection and control of discrete nozzle positions during surgery. This design allows surgeons to maintain continuous visual and manual focus on delicate procedures, thereby enhancing operational precision, reducing surgeon fatigue, and minimizing inadvertent movements that could disrupt accurate retinal sheet placement.

In another aspect, the magnetic coupler includes a mechanical limit designed to prevent over-retraction of the nozzle. This built-in safety feature ensures consistent and controlled nozzle positioning, protects the injector assembly from damage, and maintains the integrity and precise alignment of the retinal sheet during loading and transplantation, ultimately enhancing surgical reliability and procedural outcomes.

In another aspect, the method further includes visually confirming the precise placement and proper orientation of the retinal graft using a dissection microscope immediately following delivery into the subretinal space. This confirmation step ensures accurate positioning, minimizes the risk of transplantation errors, and increases the likelihood of successful graft integration and optimal restoration of visual function.

In another aspect, the injector housing and associated components are thoroughly sterilized prior to the surgical procedure. Sterilization may include autoclaving, ethylene oxide (EtO) treatment, or other validated methods appropriate for maintaining the biocompatibility and structural integrity of the components. This sterilization process ensures patient safety, reduces infection risk, and complies with stringent surgical standards.

In another aspect, the linear actuator 14 is a miniature linear actuator. In a refinement, the linear actuator 14 has a no-load maximum travel speed of from about 5 mm/s to about 20 mm/s, and in a further refinement about 10 mm/s, when operated at an input voltage of about 6 V. In a particularly useful refinement, the linear actuator 14 is a PQ12-100-6-R linear actuator (Actuonix Motion Devices Inc., Victoria, BC, Canada). Other linear actuators having comparable travel, force, and form factor characteristics may also be used.

In another aspect, the motorized retinal sheet injector 10 includes one or more travel limit features configured to constrain the range of motion of the nozzle 28 between the retracted (“Min”) and fully extended (“Max”) positions. In a refinement, the travel limit features comprise hardcoded software limits implemented in firmware executed by the microcontroller 36, physical travel constraints established by a neck region of the handheld injector housing 12, or both. In a further refinement, the magnetic coupler 22 comprises a first magnet associated with the linear actuator 14 and a second magnet associated with the core shaft 20, the first and second magnets being configured to magnetically couple during normal operation and to decouple in response to a retraction force exceeding a predetermined threshold, thereby protecting the assembly from damage due to over-retraction.

In another aspect, the handheld injector housing 12 and the microcontroller 36 are each fully enclosed within respective enclosures having sealed input/output ports and sealed wiring pass-throughs to facilitate sterilization and maintenance. In a refinement, the enclosures are configured to withstand autoclave sterilization, ethylene oxide (EtO) treatment, or both.

In another aspect, the nozzle 28 and the nozzle coupler 32 are configured as a modular, removable subassembly that is interchangeable with one or more alternative nozzle subassemblies dimensioned to accommodate retinal tissue grafts of differing size. In a refinement, the nozzle 28 is dimensioned for a retinal tissue graft having a footprint of about 0.7 mm×1.2 mm. In other refinements, the nozzle 28 is dimensioned for a retinal tissue graft having a footprint in a range of from about 0.3 mm×0.5 mm to about 2 mm×4 mm.

In another aspect, the motorized retinal sheet injector 10 is dimensioned for handheld use. In a refinement, the motorized retinal sheet injector 10 has an overall length of from about 100 mm to about 170 mm, and in a further refinement about 133.5 mm. In a refinement, the linear actuator 14 has a length of about 89.5 mm. In a refinement, the neck region of the handheld injector housing 12 has a length of about 45 mm. In a refinement, the nozzle 28 has an exposed length distal of the housing of about 20 mm and an outer diameter of about 2 mm. As one of skill in the art will appreciate, these dimensions are exemplary; the dimensions of the device may be scaled to accommodate different surgical applications, including human surgical use.

In another aspect, the motorized retinal sheet injector system further includes a visual status indicator in electrical communication with the microcontroller 36 and configured to display a visual indication of a current position state of the nozzle 28. In a refinement, the visual status indicator includes a multi-color light-emitting diode (LED), such as an RGB LED, which is configured to display a first color when the nozzle 28 is in the Min position, a second color when the nozzle 28 is in the Mid position, and a third color when the nozzle 28 is in the Max position.

In another aspect, the foot pedal interface comprises three discrete foot pedals 44, 46, 48 corresponding respectively to the Min, Mid, and Max positions. In a refinement, each of the three foot pedals 44, 46, 48 is visually distinguishable from the other two foot pedals, for example by color coding or by labeling, to facilitate selection during a surgical procedure without diverting visual attention from the operative field.

In another aspect, the microcontroller 36 includes an Arduino-based microcontroller. In a refinement, the microcontroller 36 includes an Arduino Nano microcontroller. The microcontroller 36 is programmed to receive inputs from the foot pedal interface and to drive the linear actuator 14 to the Min, Mid, and Max positions according to a calibrated displacement profile that provides consistent nozzle advancement and retraction speeds across operations.

In another aspect, the nozzle 28 is fabricated by a ferrule technique in which a heat-shrinkable fluorinated ethylene propylene (FEP) tube is loosely fitted over the mandrel 16 and heated to yield a conformal sleeve over the mandrel 16. The conformal fit and the smooth interior surface of the FEP sleeve generate hydrostatic forces that assist in retaining a retinal sheet within the nozzle 28 during loading, manipulation, and delivery, thereby reducing the likelihood of inadvertent ejection or leakage of the retinal sheet prior to placement in the subretinal space.

In another aspect, the motorized retinal sheet injector 10 is configured to deliver a payload selected from a sheet of retinal tissue, a retinal organoid, a co-graft of neural retina and retinal pigment epithelium, a matrix of cells or tissue, a synthetic substrate, a drug depot, a gene therapy depot, a liquid bolus, and combinations thereof. In a refinement, the payload includes a co-graft of human embryonic stem cell (hESC)-derived neural retina and hESC-derived retinal pigment epithelium.

In another aspect, the motorized retinal sheet injector 10 is used in surgical training applications, in research applications for the development and testing of cell replacement therapies, in clinical trials evaluating retinal transplantation techniques, or in combinations thereof.

In another aspect, the motorized retinal sheet injector 10 includes a software-implemented limit that constrains motion of the linear actuator 14. The software-implemented limit may be coded as one or more software-implemented limit values stored in firmware of the microcontroller 36. In a refinement, the software-implemented limit includes a minimum displacement software-implemented limit, an intermediate displacement software-implemented limit, and a maximum displacement software-implemented limit. Each software-implemented limit is selected to constrain travel of the nozzle 28 to within a corresponding range between the Min position, the Mid position, and the Max position.

In another aspect, the magnetic coupler 22 is configured to decouple from the core shaft 20 in response to over-retraction. In a refinement, the magnetic coupler 22 is configured to decouple from the linear actuator 14 in response to a retraction force exceeding a predetermined threshold. The first magnet of the magnetic coupler 22 and the second magnet of the magnetic coupler 22 are configured to decouple from one another such that the magnetic coupler 22 is configured to decouple non-destructively. The magnetic coupler 22 is further configured to decouple and re-couple repeatedly without damage to the magnetic coupler 22 or to the core shaft 20. Where the magnetic coupler 22 is configured to decouple in response to a retraction force exceeding the predetermined threshold, the linear actuator 14 is protected from over-travel.

In another aspect, the motorized retinal sheet injector 10 is sterilizable by autoclaving and ethylene oxide treatment. The combination of autoclaving and ethylene oxide treatment may be used sequentially or alternatively. Autoclaving and ethylene oxide treatment are each compatible with the autoclavable resin from which the handheld injector housing 12 and other 3D-printed components are fabricated. In a refinement, sterilization by autoclaving and ethylene oxide treatment is performed prior to each use. In a further refinement, autoclaving and ethylene oxide treatment cycles are selected to maintain structural integrity of components made from the autoclavable resin. Sterilization by autoclaving and ethylene oxide treatment ensures that the handheld injector housing 12 remains sterile during a surgical procedure.

In another aspect, the nozzle 28 and the nozzle coupler 32 are provided as a modular subassembly. The modular subassembly is removable and replaceable without disassembly of the handheld injector housing 12. A first modular subassembly may be exchanged for a second modular subassembly to accommodate retinal tissue grafts of differing size. Each modular subassembly may be provided pre-sterilized in a sealed package. In a refinement, the modular subassembly is keyed to the mandrel anchor point 24 such that incorrect orientation of the modular subassembly is prevented. The modular subassembly approach permits a single motorized retinal sheet injector 10 to be configured for a range of graft sizes by selection of an appropriate modular subassembly.

In another aspect, the print-in-place assembly is fabricated as a unitary print-in-place assembly using the autoclavable resin. The print-in-place assembly minimizes the number of post-fabrication assembly steps. In a refinement, the print-in-place assembly includes the handheld injector housing 12, the core shaft 20, and the mandrel anchor point 24, all fabricated together as the print-in-place assembly using the autoclavable resin. The autoclavable resin is selected to withstand repeated autoclave cycles without degradation. In a further refinement, the autoclavable resin is a biocompatible autoclavable resin suitable for medical use, such as a BioMed-class autoclavable resin. The autoclavable resin permits the print-in-place assembly to be sterilized prior to a surgical procedure and reused. The print-in-place assembly fabricated using the autoclavable resin therefore provides a low-cost, sterilizable structural backbone for the motorized retinal sheet injector 10.

In another aspect, a method of using the motorized retinal sheet injector 10 includes an actuating step. The actuating step includes actuating the foot pedal interface to drive the linear actuator 14. During the actuating step, the microcontroller 36 receives an input signal from the foot pedal interface and, in response to the actuating step, drives the linear actuator 14 to a selected position among the Min, Mid, and Max positions. In a refinement, the actuating step includes a Min actuating step, a Mid actuating step, and a Max actuating step, each actuating step corresponding to a respective foot pedal of the foot pedal interface. The current position state of the nozzle 28 may be observed via the visual status indicator during each actuating step.

In another aspect, loading the retinal sheet into the nozzle 28 is performed on a sterile surface. The sterile surface may be the sterile working surface of a sterile dish, a sterile drape, or a sterile loading platform. The retinal sheet is positioned on the sterile surface and oriented for a desired polarity prior to loading into the nozzle 28. The desired polarity refers to a relative orientation of the retinal sheet, with the desired polarity selected such that a photoreceptor side and a retinal pigment epithelium side of the retinal sheet are positioned to match a target subretinal orientation. The sterile surface and the desired polarity are confirmed under visual magnification before the actuating step. Maintaining the retinal sheet on the sterile surface and confirming the desired polarity reduces the risk of contamination and improper graft placement.

In another aspect, the visual status indicator is configured to indicate the current position state of the nozzle 28. The current position state corresponds to the Min, Mid, or Max position. The visual status indicator may display a first color when the current position state is the Min position, a second color when the current position state is the Mid position, and a third color when the current position state is the Max position. An operator may confirm the current position state of the nozzle 28 by reference to the visual status indicator before, during, and after each actuating step. In a refinement, the current position state is updated by the microcontroller 36 in response to feedback from a position sensor associated with the linear actuator 14, and the visual status indicator is driven by the microcontroller 36 to reflect the updated current position state.

In another aspect, the handheld injector housing 12 includes sealed input/output ports and sealed wiring pass-throughs configured to permit sterilization. The sealed input/output ports are configured to permit electrical connection between the microcontroller 36 and the foot pedal interface while preventing ingress of fluid. The sealed wiring pass-throughs are configured to permit wiring to extend between the handheld injector housing 12 and the linear actuator 14 while maintaining a sealed enclosure. The handheld injector housing 12 is configured to permit autoclaving and ethylene oxide treatment without compromising the sealed input/output ports or the sealed wiring pass-throughs. In a refinement, the sealed wiring pass-throughs are configured to permit replacement of wiring without re-sealing the handheld injector housing 12.

In another aspect, the predetermined threshold is selected to be greater than retraction forces encountered during normal operation. The predetermined threshold may be set in firmware of the microcontroller 36 or established by a magnetic strength of the magnetic coupler 22. The predetermined threshold is selected such that the magnetic coupler 22 decouples before damage to the linear actuator 14, the core shaft 20, the nozzle coupler 32, the nozzle 28, or the mandrel 16. In a refinement, the predetermined threshold is adjustable. The size of the retinal tissue grafts may be selected from among small retinal tissue grafts, intermediate retinal tissue grafts, and large retinal tissue grafts, and the predetermined threshold is correspondingly adjusted to suit the size of the retinal tissue grafts. The retinal tissue grafts of differing size are accommodated by the modular subassembly described above.

In another aspect, the no-load maximum travel speed of the linear actuator 14 is from about 5 mm/s to about 20 mm/s. In a refinement, the no-load maximum travel speed is about 10 mm/s. The no-load maximum travel speed is selected based on the input voltage applied to the linear actuator 14. The input voltage may be from about 3 V to about 12 V, and in a refinement the input voltage is about 6 V. The Arduino-based microcontroller drives the linear actuator 14 at the no-load maximum travel speed corresponding to the input voltage, in accordance with the calibrated displacement profile. The calibrated displacement profile defines, for each of the Min, Mid, and Max positions, a target displacement and an advancement speed of the linear actuator 14 that does not exceed the no-load maximum travel speed. The Arduino-based microcontroller may be re-programmed to modify the calibrated displacement profile, the input voltage, or both. In a further refinement, the Arduino-based microcontroller is an Arduino Nano programmed with the calibrated displacement profile.

In another aspect, the microcontroller 36 is configured to control the linear actuator 14 according to input signals from the foot pedal interface. The microcontroller 36 controls the linear actuator 14 to displace the nozzle 28 to the retracted position, the Mid position, or the fully extended position. The fully extended position corresponds to the Max position; the Mid position corresponds to an intermediate position; and the retracted position corresponds to the Min position. In a refinement, the microcontroller 36 is programmed to control the linear actuator 14 to drive the nozzle 28 directly to the fully extended position, directly to the Mid position, or directly to the retracted position in response to a corresponding foot pedal actuating step. The microcontroller 36 is further configured to control the linear actuator 14 to maintain the nozzle 28 at the fully extended position, the Mid position, or the retracted position until a subsequent input signal is received.

In another aspect, the retinal sheet may include a neural retina, a retinal pigment epithelium, or a co-graft including both a neural retina and a retinal pigment epithelium. The co-graft of neural retina and retinal pigment epithelium may be derived from human pluripotent stem cells. The neural retina of the co-graft may include photoreceptor progenitor cells. In a refinement, the co-graft is a co-graft of fetal neural retina and fetal retinal pigment epithelium. In another refinement, the co-graft is a co-graft of hESC-derived neural retina and hESC-derived retinal pigment epithelium. In a further refinement, the neural retina is provided as a sheet and the retinal pigment epithelium is provided as a sheet, and the neural retina sheet and the retinal pigment epithelium sheet are co-grafted together to form the co-graft.

In another aspect, the foot pedal interface enables hands-free selection of discrete nozzle positions. The hands-free selection of discrete nozzle positions is performed by actuation of foot pedals corresponding to the discrete nozzle positions. In a refinement, the discrete nozzle positions include the Min position, the Mid position, and the Max position, and the hands-free selection of discrete nozzle positions enables an operator to confirm graft placement and central alignment of the retinal sheet without manual manipulation. The mechanical limit of the magnetic coupler 22 cooperates with the hands-free selection of discrete nozzle positions to prevent over-retraction. The mechanical limit may include a physical mechanical limit, a magnetic mechanical limit, or both. Confirmation of graft placement and central alignment may be performed visually, for example by a dissection microscope, after each hands-free selection of one of the discrete nozzle positions. The mechanical limit, the discrete nozzle positions, the hands-free selection, the graft placement confirmation, and the central alignment verification together provide a controlled, repeatable transplantation workflow.

In another aspect, the payload delivered by the motorized retinal sheet injector 10 may include a drug depot. The drug depot may be a sustained-release drug depot, a biodegradable drug depot, or a non-biodegradable drug depot. The drug depot is loaded into the nozzle 28 and released from the distal end of the motorized retinal sheet injector 10 into the subretinal space. The distal end includes the nozzle tip 30. In a refinement, the drug depot is positioned at the distal end of the nozzle 28 prior to the actuating step, and the actuating step displaces the drug depot from the distal end into the subretinal space. The distal end of the motorized retinal sheet injector 10 may be shaped to facilitate atraumatic insertion. The drug depot may be combined with a retinal sheet payload at the distal end, such that the drug depot and the retinal sheet are delivered together from the distal end.

In another aspect, the nozzle 28 includes heat-shrink tubing fitted over the mandrel 16. The heat-shrink tubing may be fluorinated ethylene propylene heat-shrink tubing. The heat-shrink tubing is heat-conformed to the mandrel 16 to provide a smooth, conformal sleeve. In a refinement, the mandrel 16 is fabricated from stainless steel. The stainless steel mandrel provides structural rigidity and biocompatibility. The stainless steel and the heat-shrink tubing together cooperate to define a low-friction interface that facilitates loading and delivery of the retinal sheet. In a further refinement, the stainless steel includes medical-grade stainless steel selected for biocompatibility, and the heat-shrink tubing is selected for chemical compatibility with the stainless steel.

Additional details are found in Lim J, Ung F, Malhotra S, Diaz J C, Hamilton A, Chen C, Tang W C, Seiler M J, Browne A W. A Novel, Low-Cost, 3D-Printed Motorized Injector for Retinal Sheet Transplantation. Bioengineering (Basel). 2026 Feb. 6; 13(2): 188. doi: 10.3390/bioengineering13020188. PMID: 41749728; PMCID: PMC 12937642 and its Supplementary Materials; the entire disclosures of which are hereby incorporated by reference.

The following examples illustrate the various aspects of the present invention. Those skilled in the art will recognize many variations that are within the spirit of the present invention and scope of the claims.

### 1. Methods

### Design of Motorized Injector System

The overall assembly and operation of the system are illustrated in FIG. 2. The motorized injector was designed using a print-in-place approach that minimized post-processing assembly.

The device integrated commercially available components with custom-machined metal, plastic, and 3D-printed parts to reduce costs while ensuring high performance. The primary components—including the injector case, core shaft, and mandrel actuator point—were 3D-printed with a Formlabs 3B (Formlabs, MA) stereolithographic printer using BioMed resin which is biocompatible and autoclavable. An off-the-shelf linear actuator, typical of hobbyist applications, was magnetically coupled to the core shaft via a custom 3D-printed magnetic coupler, ensuring a secure connection with a limit stop to prevent over-retraction beyond the fully retracted state and damage to the system assembly.

The nozzle assembly was actuated through controlled, incremental displacements transmitted from the linear actuator to the core shaft. The nozzle coupler slides along a fixed stainless-steel mandrel embedded within the mandrel anchor point. The nozzle was fabricated with a conventional ferrule technique, in which a heat-shrinkable fluorinated ethylene propylene (FEP) nozzle was loosely fitted over the mandrel and heated, yielding a conformally fitted nozzle optimized for injectable organoids. Stainless steel was selected as the material for the mandrel for its superior rigidity and biocompatibility compared to alternatives.

Both the injector and the electronic units were hermetically enclosed, with sealed I/O ports and wiring to allow for sterilization and maintenance. Control of the injector was achieved with three actuator pedals as input to an Arduino microcontroller, via a foot pedal interface that provided three positional commands—labeled “Min,” “Mid,” and “Max”—to enable hands-free operation during surgical procedures. The system was tested and calibrated to ensure consistent nozzle advancement and retraction speeds.

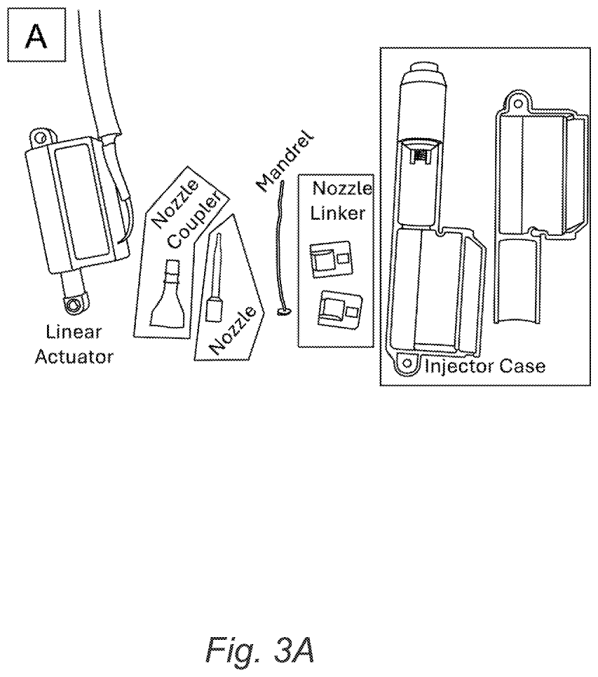

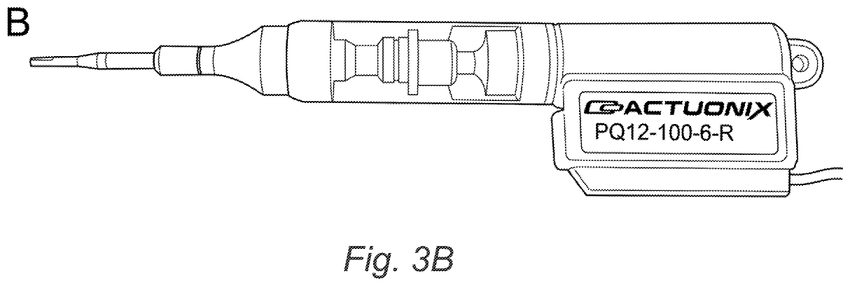

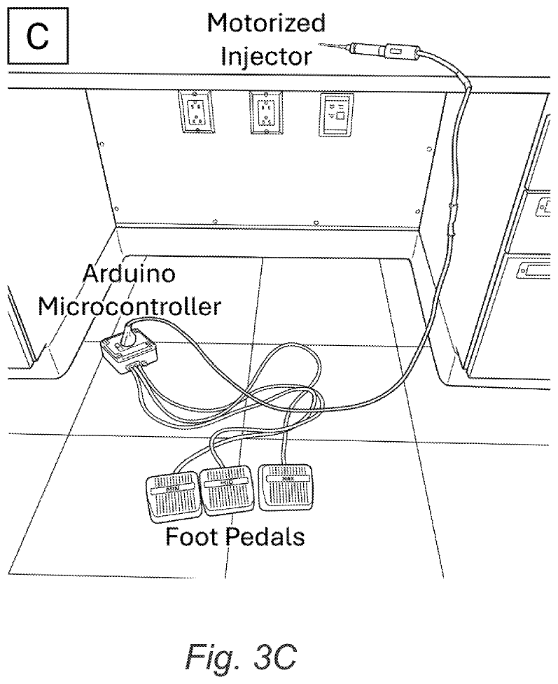

The manufactured components and assembled system are shown in FIG. 3.

### Measurement of Injector Tip Movement

To quantify and compare the instrument nozzle tip movements of both the manual and motorized instruments, we recorded videos of the motions of the nozzle tip's movement during operation. Four participants were tasked with operating both the manual and automatic injectors by fully extending and retracting the mandrel three times. Each of the participants was asked to stabilize their hand on a wrist rest throughout the trials while video was recorded with an iPhone 14 Pro on a tripod (Apple Inc., Cupertino, CA, USA). Frame-by-frame analyses were performed on the recordings using Tracker, a free video analysis and modeling tool developed by Open Source Physics. 18 The position of the tip of the mandrel was plotted in a Cartesian plane. The variances in position were calculated and an F-test for equality of variances was performed to determine statistical significance. The average and maximum velocities were also calculated and compared between devices for each participant.

### Accuracy of Retinal Sheet Placement

We evaluated, in vitro, the accuracy in placing a simulated sheet of retina on a target location. To do this we embedded pieces of 1×2 mm paper in gelatin (Knox original unflavored gelatin) to a depth of 20 mm beneath the gelatin surface. Smaller pieces of paper (0.7×1.2 mm) were then loaded into the manual or motorized injector nozzles and participants were tasked with placing and injecting the smaller piece of paper onto the larger piece of paper embedded in gelatin. All tasks were performed by participants using a dissection microscope (Leica MZ9.5) and repeated by each participant three times for each injector. The area overlap between the larger and smaller pieces of paper were quantified from photographs. The percentage of area overlap was used to represent placement accuracy and the cumulative results were plotted.

### 2. Results

The manufactured components and fully assembled motorized injector and operational system are shown in (FIG. 3).

### Measurement of Injector Tip Movement

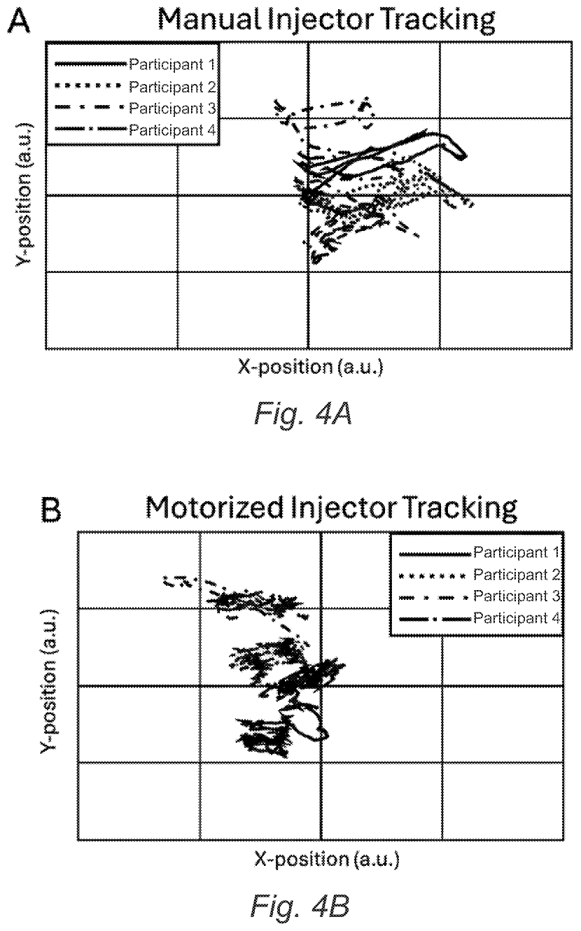

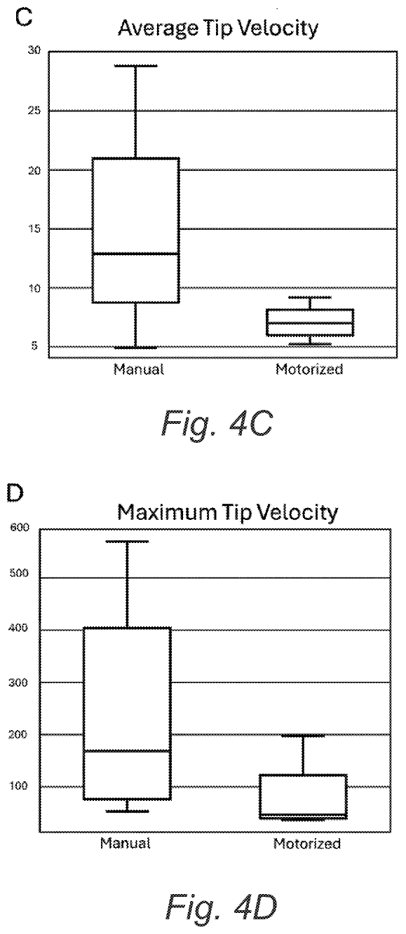

FIG. 4 shows the tracked tip trajectories and velocity distributions for both the manual and the motorized systems. Panels A and B show the Cartesian plots of tip positions for the manual and motorized injectors, respectively, overlaying the results from all participants. Notably, manual trials exhibited roughly threefold greater dispersion in tip positions compared to motorized trials (all F-tests, p<10-11). Quantification of average and maximum tip velocities (FIG. 4C-D) demonstrated lower velocity variability for the motorized system, while tip velocity of the manual system was about twice that of the motorized one. However, differences in tip velocities were not statistically significant (one-tailed t-tests, p>0.1).

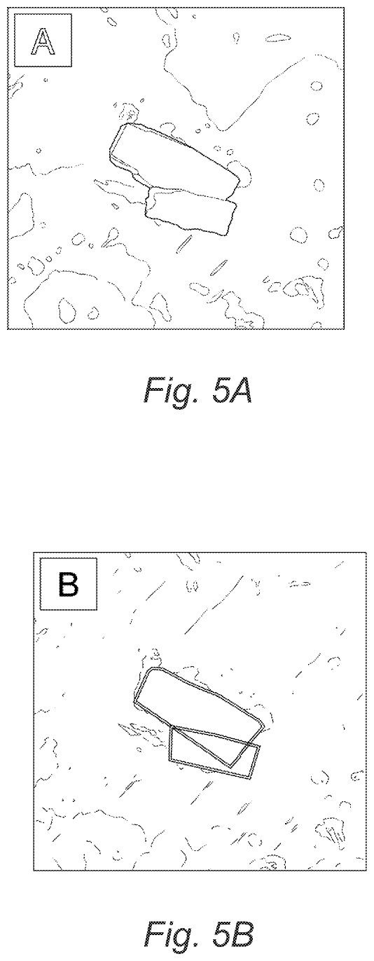

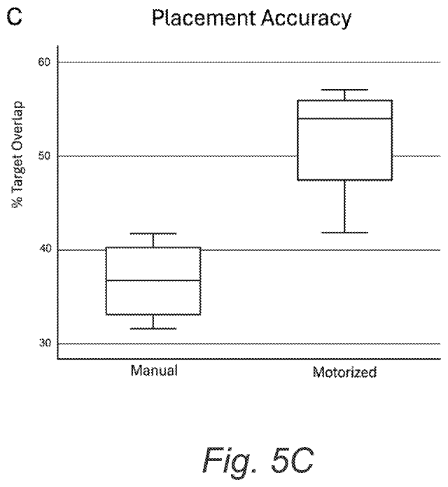

FIG. 5 summarizes placement accuracy across all four participants. A representative trial and the method for calculating percent target overlap by dividing the overlapping area of the injectable by its total area is illustrated in FIG. 5A-B. Overall, the motorized injector yielded significantly higher accuracy than the manual injector, with average accuracies of approximately 52% (95% confidence interval: 44.9-58.7%) and 37% (95% confidence interval: 19.42-54.11%), respectively (one-way ANOVA, F=13.75, p=0.00999, n=8, η²=0.696, Cohen's d=2.62) (FIG. 5C). Notably, occasional manual trials recorded 0% accuracy when the target was completely missed.

### 3. Discussion

We developed a 3D printed, motorized injector designed to enhance the precision of subretinal sheet transplantation by minimizing operator-induced uncertainty. The device's biocompatible, sterilizable construction and foot pedal-controlled linear actuation allowed for more controlled nozzle movements compared to the manual injector. Our stability trials showed that the motorized injector produced significantly lower variability in tip positioning for 4 different operators, suggesting statistical significance in improved control. Although differences in tip velocities between the two injectors were not statistically significant, the overall trend toward lower velocities with the motorized system might contribute to enhanced precision and less anatomical trauma to graft and host during subretinal placement.

In simulated retinal sheet placement tasks, the motorized injector achieved a substantially higher average placement accuracy—approximately 52% versus 37% for the manual injector. Notably, instances of complete misplacement were only observed with the manual method. These findings indicated that the motorized injector might reduce complications associated with tissue misplacement and inadvertent damage during transplantation.

Despite these promising results, however, there are several limitations. The study was conducted with only four participants, and the in vitro model may not fully replicate in vivo conditions. Additionally, operator variability and learning effects were not comprehensively addressed. Finally, the clinical relevance of the measured parameters requires further validation in animal models.

### 4. CONCLUSION

Our findings demonstrate that the motorized injector significantly improves placement accuracy and operational stability compared to the manual injector. By reducing undesired hand tremor effects and enhancing control, this system shows promise for improving the outcomes of subretinal sheet transplantation. Future work should focus on validating these results in vivo.

While exemplary aspects are described above, it is not intended that these aspects describe all possible forms of the invention. Rather, the words used in the specification are words of description rather than limitation, and it is understood that various changes may be made without departing from the spirit and scope of the invention. Additionally, the features of various implementing aspects may be combined to form further aspects of the invention.

### REFERENCES

1. Rachitskaya A, Yuan A, Davidson S, et al. Computer-Assisted Immersive Visual Rehabilitation in Argus II Retinal Prosthesis Recipients. Ophthalmol Retina. 2020; 4(6): 613-619. doi: 10.1016/j.oret.2019.11.007
2. Humayun M S, Lee S Y. Advanced Retina Implants. Ophthalmol Retina. 2022; 6(10): 899-905. doi: 10.1016/j.oret.2022.04.009
3. Gogliettino A R, Madugula S S, Grosberg L E, et al. High-Fidelity Reproduction of Visual Signals by Electrical Stimulation in the Central Primate Retina. J Neurosci Off J Soc Neurosci. 2023; 43(25): 4625-4641. doi: 10.1523/JNEUROSCI.1091-22.2023
4. Muqit M M K, Le Mer Y, Olmos de Koo L, Holz F G, Sahel J A, Palanker D. Prosthetic Visual Acuity with the PRIMA Subretinal Microchip in Patients with Atrophic Age-Related Macular Degeneration at 4 Years Follow-up. Ophthalmol Sci. 2024; 4(5): 100510. doi: 10.1016/j.xops.2024.100510
5. Schwartz S D, Tan G, Hosseini H, Nagiel A. Subretinal Transplantation of Embryonic Stem Cell-Derived Retinal Pigment Epithelium for the Treatment of Macular Degeneration: An Assessment at 4 Years. Invest Ophthalmol Vis Sci. 2016; 57(5): ORSFc1-9. doi: 10.1167/iovs.15-18681
6. Kashani A H, Lebkowski J S, Rahhal F M, et al. A bioengineered retinal pigment epithelial monolayer for advanced, dry age-related macular degeneration. Sci Transl Med. 2018; 10(435): eaao4097. doi: 10.1126/scitranslmed.aao4097
7. Radtke N D, Aramant R B, Petry H M, Green P T, Pidwell D J, Seiler M J. Vision Improvement in Retinal Degeneration Patients by Implantation of Retina Together with Retinal Pigment Epithelium. Am J Ophthalmol. 2008; 146(2): 172-182.el. doi: 10.1016/j.ajo.2008.04.009
8. Seiler M J, Aramant R B. Cell replacement and visual restoration by retinal sheet transplants. Prog Retin Eye Res. 2012; 31(6): 661-687. doi: 10.1016/j.preteyeres.2012.06.003
9. Seiler M J, Lin R E, Mclelland B T, et al. Vision Recovery and Connectivity by Fetal Retinal Sheet Transplantation in an Immunodeficient Retinal Degenerate Rat Model. Investig Opthalmology Vis Sci. 2017; 58(1): 614. doi: 10.1167/iovs.15-19028
10. Lin B, McLelland B T, Mathur A, Aramant R B, Seiler M J. Sheets of human retinal progenitor transplants improve vision in rats with severe retinal degeneration. Exp Eye Res. 2018; 174: 13-28. doi: 10.1016/j.exer.2018.05.017
11. Lin B, McLelland B T, Aramant R B, et al. Retina Organoid Transplants Develop Photoreceptors and Improve Visual Function in RCS Rats With RPE Dysfunction. Investig Opthalmology Vis Sci. 2020; 61(11): 34. doi: 10.1167/iovs.61.11.34
12. McLelland B T, Lin B, Mathur A, et al. Transplanted hESC-Derived Retina Organoid Sheets Differentiate, Integrate, and Improve Visual Function in Retinal Degenerate Rats. Investig Opthalmology Vis Sci. 2018; 59(6): 2586. doi: 10.1167/iovs.17-23646
13. Lin B, Singh R K, Seiler M J, Nasonkin I O. Survival and Functional Integration of Human Embryonic Stem Cell-Derived Retinal Organoids After Shipping and Transplantation into Retinal Degeneration Rats. Stem Cells Dev. 2024; 33(9-10): 201-213. doi: 10.1089/scd.2023.0257
14. Watari K, Yamasaki S, Tu H Y, et al. Self-organization, quality control, and preclinical studies of human iPSC-derived retinal sheets for tissue-transplantation therapy. Commun Biol. 2023; 6(1): 164. doi: 10.1038/s42003-023-04543-5
15. Iraha S, Tu H Y, Yamasaki S, et al. Establishment of Immunodeficient Retinal Degeneration Model Mice and Functional Maturation of Human ESC-Derived Retinal Sheets after Transplantation. Stem Cell Rep. 2018; 10(3): 1059-1074. doi: 10.1016/j.stemcr.2018.01.032
16. Assawachananont J, Mandai M, Okamoto S, et al. Transplantation of Embryonic and Induced Pluripotent Stem Cell-Derived 3D Retinal Sheets into Retinal Degenerative Mice. Stem Cell Rep. 2014; 2(5): 662-674. doi: 10.1016/j.stemcr.2014.03.011
17. Matsuyama T, Tu H Y, Sun J, et al. Genetically engineered stem cell-derived retinal grafts for improved retinal reconstruction after transplantation. iScience. 2021; 24(8): 102866. doi: 10.1016/j.isci.2021.102866
18. Brown D. Tracker Video Analysis and Modeling Tool. https://opensourcephysics.github.io/tracker-website/

1. A motorized retinal sheet injector for delivering retinal material into a subretinal space, comprising:
a handheld injector housing;
a linear actuator at least partially housed within the handheld injector housing;
a mandrel fixed relative to the handheld injector housing and extending along a longitudinal axis;
a core shaft;
a magnetic coupler operably connecting the linear actuator to the core shaft;
a mandrel anchor point fixed to the core shaft;
a nozzle disposed coaxially with the mandrel and configured to slide along the mandrel when actuated, the nozzle having a nozzle tip;
a nozzle coupler configured to connect the mandrel anchor point to the nozzle, such that actuation force from the linear actuator is transmitted to the nozzle via the core shaft;
a microcontroller in electrical communication with the linear actuator; and
a foot pedal interface in electrical communication with the microcontroller, wherein the microcontroller is configured to control the linear actuator based on input from the foot pedal interface, and wherein the linear actuator is operable to position the nozzle in at least three discrete positions comprising a retracted position (“Min”), an intermediate position (“Mid”), and a fully extended position (“Max”).

2. The injector of claim 1, wherein at least one of the handheld injector housing, the magnetic coupler, the nozzle, and the nozzle coupler is at least partially fabricated using a 3D printing process with a biocompatible, sterilizable resin.

3. The injector of claim 1, wherein the nozzle comprises fluorinated ethylene propylene (FEP) heat-shrink tubing conformally fitted over the mandrel to provide a sleeve for retaining a retinal sheet.

4. The injector of claim 1, wherein the mandrel is made of stainless steel to provide structural rigidity and biocompatibility.

5. The injector of claim 1, further configured for loading a retinal sheet into the nozzle, wherein the microcontroller is configured to:
maintain the nozzle in a retracted (“Min”) position while the retinal sheet is positioned on a sterile working surface under visual magnification;
advance the nozzle to an intermediate (“Mid”) position to align the nozzle tip with the retinal sheet; and
further advance the nozzle toward the fully extended position (“Max”) to engage and envelop the retinal sheet within the nozzle without compressing it.

6. The injector of claim 1, wherein the microcontroller comprises a microcontroller board configured to receive foot pedal inputs and actuate the linear actuator to the Min, Mid, and Max positions.

7. The injector of claim 1, wherein the linear actuator has a no-load maximum travel speed of from about 5 mm/s to about 20 mm/s when operated at an input voltage of about 6 V.

8. The injector of claim 1, further comprising one or more travel limit features configured to constrain a range of motion of the nozzle, the one or more travel limit features comprising at least one of: (a) a software-implemented limit executed by the microcontroller, and (b) a physical travel stop established by a neck region of the handheld injector housing.

9. The injector of claim 1, wherein the magnetic coupler comprises a first magnet associated with the linear actuator and a second magnet associated with the core shaft, the first and second magnets being configured to decouple in response to a retraction force exceeding a predetermined threshold.

10. The injector of claim 1, wherein the handheld injector housing and an enclosure of the microcontroller each include sealed input/output ports and sealed wiring pass-throughs configured to permit sterilization by at least one of autoclaving and ethylene oxide treatment.

11. The injector of claim 1, wherein the nozzle and the nozzle coupler are configured as a modular subassembly that is interchangeable with one or more alternative nozzle subassemblies dimensioned to accommodate retinal tissue grafts of differing size.

12. The injector of claim 1, further comprising a multi-color light-emitting diode in electrical communication with the microcontroller and configured to display a first color when the nozzle is in the Min position, a second color when the nozzle is in the Mid position, and a third color when the nozzle is in the Max position.

13. The injector of claim 1, wherein the microcontroller comprises an Arduino-based microcontroller programmed to drive the linear actuator to the Min, Mid, and Max positions according to a calibrated displacement profile.

14. The injector of claim 1, wherein the foot pedal interface comprises three discrete foot pedals corresponding respectively to the Min, Mid, and Max positions, and wherein each of the three foot pedals is visually distinguishable from the other two foot pedals.

15. The injector of claim 1, wherein at least the handheld injector housing, the core shaft, and a mandrel anchor point are fabricated as a print-in-place assembly using a stereolithographic 3D printing process and a biocompatible, autoclavable resin.

16. A method of treating a subject with retinal degeneration, comprising:
providing a motorized retinal sheet injector system of claim 1;
loading a retinal sheet into the nozzle;
inserting a distal end of the injector into a subretinal space of an eye of the subject;
actuating a foot pedal of the foot pedal interface to advance the nozzle over the retinal sheet via the linear actuator, magnetic coupler, and core shaft assembly;
releasing the retinal sheet into the subretinal space by retracting the nozzle over the mandrel while the mandrel maintains the retinal sheet in the subretinal space; and
withdrawing the injector while maintaining the mandrel in a fixed position.

17. The method of claim 16, wherein the retinal sheet comprises a retinal organoid derived from human pluripotent stem cells.

18. The method of claim 16, wherein the retinal degeneration is selected from the group consisting of age-related macular degeneration, retinitis pigmentosa, and Stargardt disease.

19. The method of claim 16, wherein the step of loading the retinal sheet into the nozzle comprises:
positioning the retinal sheet on a sterile surface under a microscope;
orienting the retinal sheet for desired polarity;
partially advancing the nozzle to a “Mid” position;
gently capturing the sheet within the nozzle tip; and
confirming central alignment of the sheet within the nozzle.

20. The method of claim 16, wherein the nozzle comprises a fluorinated ethylene propylene (FEP) sleeve heat-conformed over the mandrel.

21. The method of claim 16, wherein the foot pedal interface enables hands-free selection of discrete nozzle positions during surgery.

22. The method of claim 16, wherein the magnetic coupler includes a mechanical limit, and wherein the method further comprises engaging the mechanical limit to prevent over-retraction of the nozzle.

23. The method of claim 16, further comprising visually confirming graft placement using a dissection microscope.

24. The method of claim 16, wherein the injector housing and components are sterilized prior to a surgical procedure.

25. The method of claim 16, wherein the retinal sheet comprises a co-graft of neural retina and retinal pigment epithelium derived from human pluripotent stem cells.

26. The method of claim 16, further comprising observing a visual status indicator in electrical communication with the microcontroller to confirm a current position state of the nozzle during the actuating step.

27. A method of subretinal delivery, comprising:
providing the motorized retinal sheet injector of claim 1;
loading a payload into the nozzle, the payload comprising at least one of a drug depot, a gene therapy depot, a liquid bolus, a synthetic substrate, and a matrix of cells or tissue;
inserting a distal end of the injector into a subretinal space of an eye of a subject;
actuating a foot pedal of the foot pedal interface to advance the nozzle and release the payload into the subretinal space; and withdrawing the injector while maintaining the mandrel in a fixed position.
